(19) United States
(12) Patent Application Publication
(10) Pub. No.: US 2005/0129615 A1
(43) Pub. Date: Jun. 16, 2005

(54) Non-invasive system for detecting skin cancer

(76) Inventors: Jacek Rozga, Beverly Hills, CA (US); Toomas Neuman, La Jolla, CA (US)

Correspondence Address: Dorsey & Whitney LLP;Intellectual Property Department, Suite 3400, Four Embarcadero Center, San Francisco, CA, 94111-4187, US

(21) Appl. No.: 10/961,580

(22) Filed: Oct. 8, 2004

Related U.S. Application Data
(60) Provisional application No. 60/509,988, filed on Oct. 9, 2003.

(51) Int. Cl. A61K 51/00; G01N 33/574

(52) U.S. Cl. 424/1.49; 435/7.23

(57) ABSTRACT

The invention concerns methods and compositions for the diagnosis of cancer. The invention further concerns diagnostic patches which may be contacted topically with an externally accessible tissue surface for the purpose of detecting the presence or absence of cancer.

### BACKGROUND OF THE INVENTION

The skin has three layers, the epidermis, the dermis, and the subcutis. The top layer of the skin, the epidermis, is very thin and serves to protect the deeper layers of skin and the internal organs. The epidermis itself has three layers: an upper, a middle, and a bottom layer composed of basal cells. These basal cells divide to form keratinocytes, (also called squamous cells) which make a keratin. Melanocytes are also present in the epidermis. These cells produce the pigment called melanin. Melanin gives the tan or brown color to skin and helps protect the deeper layers of the skin from the harmful effects of the sun. A basement membrane comprised of extracellular matrix separates the epidermis from the deeper layers of skin.

There are a number of different types of skin cancer. Nonmelanomas (usually basal cell and squamous cell cancers) are the most common cancers of the skin. They are called nonmelanoma because they develop from skin cells other than melanocytes. They rarely spread to sites elsewhere in the body. Melanoma is a cancer that begins in cells of the melanocytic system of the skin, and metastasis of melanoma is common. Although melanoma accounts for only about 4% of all skin cancer cases, it causes most skin cancer-related deaths.

Different forms of skin cancer are characterized by the expression of a specific patterns of extracellular proteinases. Activity of serine proteinases such as u-PA and t-PA has been used for classification and prognosis of skin cancer (Maguire et al., Low levels of urokinase plasminogen activator components in basal cell carcinoma of the skin, Int J Cancer. 85(4):457-9 [2000]; Ferrier et al., High tPA-expression in primary melanoma of the limb correlates with good prognosis, Br J Cancer 83(10):1351-9 [2000]).

Increased activity of another group of proteinases (matrix metalloproteinases; MMPs) has also been described in skin cancer. Degradation of basement membranes and extracellular matrix is an essential step in skin cancer cell migration, invasion and metastasis formation. Matrix metalloproteinases and their inhibitors play a crucial role in these complex multistep processes. Skin cancer cells express a number of matrix metalloproteinase family members such as MMP- I, MMP-2, MMP-9, MMP-13, MT I-MMP and others (Dumas et al., Expression of basement membrane antigens and matrix metalloproteinases 2 and 9 in cutaneous basal and squamous cell carcinomas, Anticancer Res. 19(4B):2929-38 [1999]; Walker RA, Woolley DE, Immunolocalisation studies of matrix metalloproteinases-1, -2 and -3 in human melanoma, Virchows Arch. 435(6):574-9 [1999]; Airo Fusenig NE, Differential stromal regulation of MMP-1 expression in benign and malignant keratinocytes, J Invest Dermatol. 116(1):85-92 [2001]; Bodey et al, Matrix metalloproteinase expression in malignant melanomas: tumor-extracellular matrix interactions in invasion and metastasis, In Vivo 15(1):57-64 [2001]; Kerkela et al, Differential patterns of stromelysin-2 (MMP-10) and MT1-MMP (MMP-14) expression in epithelial skin cancers, Br J Cancer 84(5):659-69 [2001]; Papathoma et al., Role of matrix metalloproteinase-9 in progression of mouse skin carcinogenesis, Mol. Carcinog. 31(2):74-82 [2001]) as well as their tissue inhibitors TIMP-1, TIMP-2 and TIMP-3 (for review see Hofmann et al., Matrix metalloproteinases in human melanoma, J Invest Dermatol 115(3):337-44 [2000]).

Because particular skin lesions express a specific set of extracellular proteinases that can be used to develop diagnostic tools for skin cancer. For example, U.S. Pat. No. 5,324,634 describes in vitro immunoassay of MMPs complexed with tissue inhibitors of matrix metalloproteinases (TIMPs) for detecting cancer. (Zucker, Diagnostic tests measuring gelatinase/inhibitor complexes for detection of aggressive and metastatic cancer, U.S. Pat. No. 5,324,634).

Individual extracellular proteinases have unique substrate specificity. Therefore, it, possible to identify presence and activity of individual proteinases in complex mixtures (for example tumor samples) using specific substrates. A large number of these substrates, many of which are fluorogenic, are commercially available and screening of peptide libraries can help identify new and more specific substrates (Rosse et al., Rapid identification of substrates for novel proteases using a combinatorial peptide library, J Comb Chem 2(5):461-6 [2000]; Sheppeck et al, Synthesis of a statistically exhaustive fluorescent peptide substrate library for profiling protease specificity, Bioorg Med Chem Lett 10(23):2639-42 [2000]).

The present invention exploits the substrate specificity of secreted proteolytic enzymes of the extracellular matrix, such as matrix metalloproteinases, serine protease, or cathepsin, to provide a rapid and non-invasive diagnostic test for some forms of cancer, particularly skin cancers, such as melanomas.

### SUMMARY OF THE INVENTION

The present invention is directed to a patch for detecting a cancer. The patch includes a carrier layer that includes a pharmaceutically acceptable aqueous solution, which can, for example but not necessarily, be held or contained within a hydrogel or polymeric or co-polymeric matrix. The carrier layer includes a first probe (or additional second, third, or subsequent different probes) that comprises a peptide substrate of a first (second, third, or subsequent) proteolytic enzyme of interest. The proteolytic enzyme is an extracellularly secreted enzyme, the activity of which is characteristic of the cancer, for example, a skin cancer such as, but not limited to, a melanoma. The carrier layer comprises a semipermeable surface, which semipermeable surface is adapted for being contacted topically with an externally accessible tissue surface, such as the skin, the lips, the interior of the nostrils, or a mucous membrane (e.g., mucous membrane of mouth, throat, sinuses, rectum, or vagina). The probe can diffuse through the semipermeable surface of the carrier layer. If the proteolytic enzyme is present, and catalytically active, in the extracellular matrix (ECM), the peptide substrate is cleaved to produce a detectable first (or second, third, or subsequent) cleavage product, which then diffuses back into the patch through the semipermeable surface, which is permeable to it. Detection of the cleavage product indicates the presence of the cancer in the tissue.

The present invention also relates to a method of detecting a cancer, such as a melanoma or other skin cancer, in a tissue of a mammalian subject. The method involves topically applying to an externally accessible tissue surface the inventive patch. Such externally accessible tissue surfaces include the skin, the lips, the interior of the nostrils, or a mucous membrane (e.g., mucous membrane of mouth, throat, sinuses, rectum, or vagina). The first probe or the second probe, or both, are allowed to diffuse into the tissue such that, if the first proteolytic enzyme or the second proteolytic enzyme, or both, is present in the tissue, the first probe binds to the first proteolytic enzyme, the second probe binds to the second proteolytic enzyme, and enzymatic cleavage of the relevant peptide substrate results, thereby forming the first cleavage product or the second cleavage product, or both; and then the presence or absence of the first cleavage product or the second cleavage product, or both, is detected by applying suitable detection means, whereby proteolytic activity that is characteristic of the cancer is indicated and the cancer is detected thereby. Detection and quantification of the cleavage product(s) can be done by any suitable means, either directly of cleavage product in the patch, or alternatively, the cleavage product can first be extracted from the patch for detection of the cleavage product in the extract.

The inventive patches and method, embodiments of which will be further described in detail hereinbelow, provide for non-invasive and rapid diagnosis of cancers, particularly skin cancers, such as melanoma.

### BRIEF DESCRIPTION OF THE DRAWING

FIG. 1 shows a schematic representation of an embodiment of the inventive patch for detecting a skin cancer, such as a melanoma.

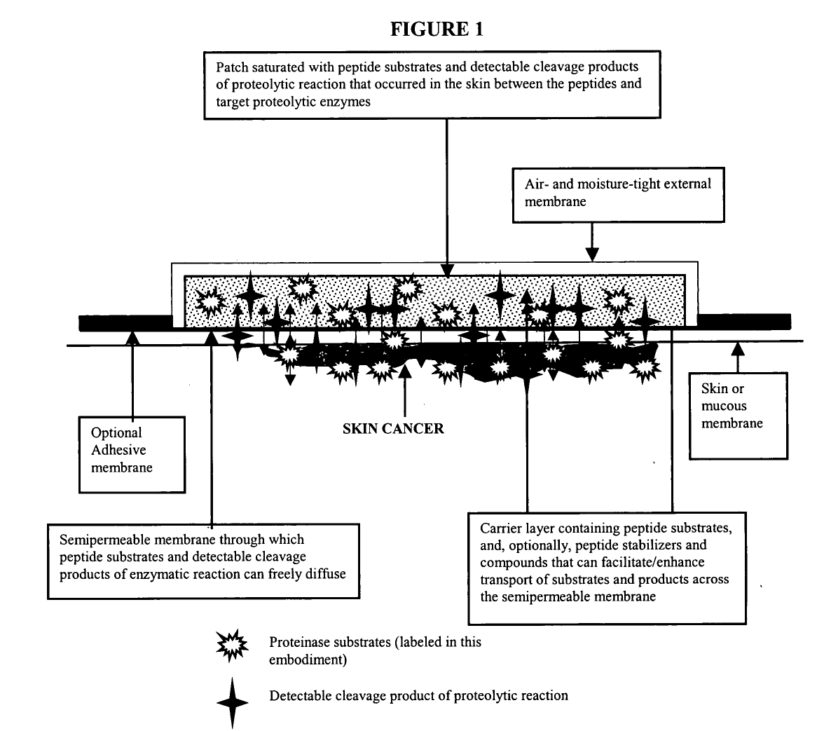

### DETAILED DESCRIPTION OF THE PREFERRED EMBODIMENTS

The patch of the present invention, which comprises the carrier layer, can employ any suitable patch technology. For example, the patch can be a matrix type transdermal or transmucosal patch. (E.g., Chien et al., U.S. Pat. Nos. 4,906,169 and 5,023,084; Cleary et al., U.S. Pat. No. 4,911,916; Teillaud et al., U.S. Pat. No. 5.605,702; Venkateshwaran et al., U.S. Pat. No. 5,783,208; Ebert et al., U.S. Pat. No. 5,460,820; Ebert et al., Transdermal delivery system with adhesive overlay and peel seal disc, U.S. Pat No. 5,662,925; Chang et al., Device for administering an active agent to the skin or mucosa, U.S. Pat. Nos. 4,849,224 and 4,983,395). The matrix of the patch can comprise a polymeric or co-polymeric basal support layer, such as an acrylic or ethylene/vinyl acetate copolymer or a polyurethane foam or cellulosic material.

In accordance with embodiments of the present invention directed particularly to transmucosal applications, a variety of pharmaceutically acceptable transmucosal patch systems are known in the art and are useful. For example, a transmucosal patch system comprising a laminated composite of, for example, an adhesive layer, a backing layer, a permeable membrane defining a reservoir containing the aqueous solution, a peel seal disc underlying the membrane, one or more heat seals, and a removable release liner. (Ebert et al., Transdermal delivery system with adhesive overlay and peel seal disc, U.S. Pat No. 5,662,925; Chang et al., Device for administering an active agent to the skin or mucosa, U.S. Pat. Nos. 4,849,224 and 4,983,395). Some useful transmucosal systems employ a non-ionic detergent along with a permeation enhancer.

These examples of useful patch technologies applicable to the present invention, are merely illustrative and are not limiting of the present invention.

In some embodiments of the inventive patch, the carrier layer also contains peptide stabilizers and/or compounds that can facilitate and/or enhance transport of substrates and products across the semipermeable surface of the carrier layer. Examples of permeation enhancers include, but are not limited to, comprising a permeation or penetration enhancer, such as polyethylene glycol monolaurate, dimethyl sulfoxide, N-vinyl-2-pyrrolidone, N-(2-hydroxyethyl)-pyrrolidone, or 3-hydroxy-N-methyl-2-pyrrolidone a bile salt or fusidate, a hydrophilic polymer, such as hydroxypropyl cellulose, hydroxypropyl methylcellulose, hydroxyethylcellulose, dextran, pectin, polyvinyl pyrrolidone, starch, gelatin, or any of a number of other polymers known to be useful for this purpose.

Preferably, the inventive patch includes an air-tight and moisture-tight external layer or membrane that keeps the pharmaceutically acceptable aqueous solution from dehydrating during use. The external layer is opposite to the semipermeable surface that is contacted with the externally accessible tissue surface.

Some embodiments of the inventive patch further comprise an adhesive layer comprising an adhesive, such as, but not limited to, polysiloxane, for adhering the patch to the externally accessible tissue surface, such as the epidermis or mucosa. Alternatively, the patch can be contacted with the externally accessible tissue surface and held in place by an overlying bandage (e.g., adhesive, gauze or other type of bandage) or suitable pressure device or means adapted for keeping the semipermeable surface of the carrier layer in contact with the tissue surface for the desired time.

Other embodiments include features include a backing layer, or peelable seal or liner that preserves operability of the patch during storage before use. Bacteriostatic agents can also be included in the pharmaceutically acceptable aqueous solution, or in the polymeric or co-polymeric matrix.

In accordance with the inventive patch, the carrier layer comprises a pharmaceutically acceptable aqueous solution, which contains one or more specific probe(s) comprising the peptide substrate, or substrates, of the proteolytic enzyme(s) of interest. In accordance with the invention, the carrier layer of the patch can contain a first, second, third, or subsequent different probe for targeting a first, second, third, or subsequent different proteolytic enzyme of interest. Preferably, but not necessarily, the peptide substrate is an oligopeptide substrate analog of the proteolytic enzyme of interest. The first, second, or subsequent proteolytic enzyme of interest can be, for example, a serine protease, a cathepsin, or a metalloproteinase.

In some preferred embodiments, the probes can be dissolved in the pharmaceutically acceptable aqueous solution. In these embodiments the probes can freely diffuse through the semipermeable membrane into the tissue.

In other embodiments, the probes are complexed to a polymeric or co-polymeric matrix within the carrier layer, and the semipermeable membrane is permeable to the first (second, third, or subsequent) proteolytic enzyme of interest, which if present, can diffuse into the patch to react with the probe(s), resulting in the production of detectable cleavage products in accordance with the invention.

In some embodiments of the inventive patch and method of detecting a cancer, such as a skin cancer, the cleavage product is detectable by interaction with a specific antibody or aptamer, or by molecular interaction with another reagent, such as a detectable nanoparticle that specifically binds the cleavage product of interest. (E.g., Nam JM, Thaxton CS, Mirkin CA, Nanoparticle-based bio-bar codes for the ultrasensitive detection of proteins, Science 301(5641):1884-6 [2003]; Cognet L, Tardin C, Boyer D, Choquet D, Tamarat P, Lounis B, Single metallic nanoparticle imaging for protein detection in cells, Proc Natl Acad Sci U S A. 2003 Sep 30;100(20):11350-11355 [2003]).

An “aptamer” is an oligonucleotide, e.g., a DNA or a RNA molecule, that binds to a specific molecular target, such as a protein or metabolite. An aptamer can be synthesized by known techniques or can be obtained commercially. (E.g., Hamaguchi, N. et al., Apatamer beacons for the detection of proteins, Analytical Biochemistry 294:126-131 [2001]).

“Antibodies” include whole antibodies, and antibody fragments, with a specific target-binding capability of interest, i.e., antigen-specific or hapten-specific targeting ligands. Antibody fragments include, for example Fab, Fab′, F(ab′)₂, or F(v) fragments. Antibodies can also be polyclonal or monoclonal antibodies. Antibodies also include antigen-specific or hapten-specific targeting ligands complexed with linker moieties to a carrier molecule.

In other embodiments of the inventive patch and method, the probe further comprises a detectable label, such that after cleavage of the oligopeptide substrate analog by the proteolytic enzyme of interest, a cleavage product is formed that bears the detectable label. In accordance with these embodiments, the label can be, but is not limited to, a fluorochrome, a radioisotope, or a stable (i.e., non-radioactive) isotope, as long as the substrate is synthesized with the label placed in a metabolically suitable location in the structure of the substrate, i.e., a location where enzymatic cleavage results in the isotopic label being sequestered in the cleavage product.

Usefully, fluorochromes can be comprised in intramolecularly-quenched fluorescence probes. Methods of making intramolecularly-quenched fluorescence probes and fluorescence detection that are useful in practicing the present invention are known. (E.g., Weissleder, et al., Intramolecularly-quenched near infrared fluorescent probes, U.S. Pat. No. 6,083,486; Beekman, B., et al., Convenient fluorometric assay for matrix metalloproteinase activity and its application in biological media, 1996. FEBS Lett 390:221 [1996]; Beekman, B., et al., Highly increased levels of active stromelysin in rheumatoid synovial fluid determined by a selective fluorogenic assay, 1997. FEBS Lett 418:305 [1997]; Ntziachristos V, Bremer C, Weissleder, R, Fluorescence Imaging with near-infrared light: new technological advances that enable in vivo molecular imaging, Eur Radiol 13:195-208 [2003]; Mahmood U, Tung CH, Bogdanov A Jr, Weissleder R., Near-infrared optical imaging of protease activity for tumor detection, Radiology 213(3):866-70 [1999]; Bremer C, Bredow S, Mahmood U, Weissleder R., Tung CH, Optical imaging of matrix metalloproteinase-2 activity in tumors: feasibility study in a mouse model, Radiology 221(2):523-9 [2001]; Bremer C, Tung CH, Weissleder R., In vivo molecular target assessment of matrix metalloproteinase inhibition, Nat Med. 7(6):743-8, Comment on 655-6 [2001];.Ntziachristos V, Tung CH, Bremer C, Weissleder R., Fluorescence molecular tomography resolves protease activity in vivo, Nat Med 8(7):757-60 [2002]; Tung CH, Mahmood U, Bredow S, Weissleder R., In vivo imaging of proteolytic enzyme activity using a novel molecular reporter, Cancer Res 60(17):4953-8 [2000]; Simonetti, Lucarini G, Brancorsini D, Nita P, Bernardini ML, Biagini G, Offidani A., Immunohistochemical expression of vascular endothelial growth factor, matrix metalloproteinase 2, and matrix metalloproteinase 9 in cutaneous melanocytic lesions, Cancer 95(9):1963-70 [2002]). An enzymatic “fingerprint” or profile of a particular tumor type can also be obtained in accordance with the inventive method.

In some embodiments of the inventive method, detecting the presence or absence of the first cleavage product or the second cleavage product, or both, is performed by applying detection means (e.g., fluorescence, radiation, nuclear magnetic resonance, immuno- or other detection means) directly to the patch. In other embodiments, the cleavage products are extracted from the patch to obtain an extract, and suitable detection means are applied to the extract.

Suitable methods for detecting the presence (or absence) of fluorescence, include but are not limited to, spectrofluorometry, fluorescence resonance energy transfer (FRET) and capillary electrophoresis with fluorescence detection means. Direct detection of the product in the patch itself can be accomplished by illumination of the patch and detection of emitting light by the naked eye or by using an array of optical devices utilizing regular, infrared, near-infrared and/or ultraviolet light, depending on the optical properties of fluorogenic substrates used in the patch.

Useful radioisotopic labels include, but are not limited to ³⁵S, ¹⁴C, or ³H, which are detectable using appropriate radiation detecting means. Alternatively, a stable isotope, such as but not limited to ¹³C, ²H, 17O or 18O can be employed as the label. Detection of stable isotopes is typically accomplished with techniques such as mass spectroscopy or nuclear magnetic resonance.

To obtain an enzymatic fingerprint of a tumor, more than one differently labeled substrate must be used. For example, two or more substrates, each specific for a different proteinase and each labeled with different fluorochromes, can be employed. Cleavage products bearing different labels are identified based on their different characteristics, for example, spectral (e.g., fluorescence) or isotopic characteristics.

In accordance with the inventive method, the semipermeable surface of the patch is kept in contact with the externally accessible tissue surface for a time sufficient for diffusion of detectable amounts of cleavage product into the patch, in the event a proteolytic enzyme of interest is present. This period is partially dependent upon the detection means employed. For example, employing a radiolabel can typically provide greater sensitivity than employing antibody-based detection of the cleavage product. Generally a period of about 15 minutes to about 30 minutes is preferable, but longer periods of about one to about two hours, or longer, can be employed.

All references cited herein are incorporated in their entirety by reference.

While the invention has been described with reference to its preferred embodiments, it will be appreciated by those skilled in this art that variations can be made departing from the precise examples of the methods and compositions disclosed herein, which, nonetheless, embody the invention.

### EXAMPLES

### Example 1

### Detection of Fluorescent Products of Proteolysis Using Cellulose Membrane Patch

Experiments were performed to demonstrate that fluorogenic peptide substrates diffuse from the cellulose patch to the source of MMPs, and fluorescent products of the enzymatic reaction diffuse back to the patch and can be detected using ultraviolet (UV) light.

Electrophoresis grade agar (Sigma) in 20 mM Tris buffer, pH 7.6, 2.5 mM ZnSO₄ and 5 mM CaCl₂ was melted and poured into 60-mm Petri dishes. Next, 0.5 microliters of active MMP2 and MMP6 (concentration 0.001 micrograms/microliter, Oncogene Research Products) was injected into the agar (0, 2 and 5 mm below the surface). Buffer without enzymes was used as a control. Injection sites were covered with a cellulose membrane patch previously immersed in a solution of high affinity fluorogenic substrates for MMPs (10⁻⁵ M of DABCYL-GABA-Pro-Gln-Gly-Leu-Glu(EDANS)-Ala-Lys-NH₂ [SEQ ID NO:1]; for proteolytic product, excitation maximum is λ=340 nm and emission maximum is λ=485 nm; Calbiochem, Catalog No. 444256 and/or DABCYL-GABA-Arg-Pro-Lys-Pro-Val-Glu-Nva-Trp-Arg-Glu(EDANS)-Ala-Lys-NH [SEQ ID NO:2], Nva =Norvaline; for proteolytic product, excitation maximum is λ=360 nm and emission maximum is λ=490 nm; Calbiochem, Catalog No. 444257). Membrane patches were removed after 30, 60 and 120 minutes and fluorescence was semiquantitatively analyzed by viewing with a fluorescence microscope using a “DAPI”filter with excitation maximum 340-360 nm and emission maximum 460-490 nm. Fluorescence was detected in the patches under experimental conditions, but not in controls. As shown in Table 1, the relative fluorescence indirectly correlated with the distance of the substrate from the patch at the agar surface and the incubation time, which is consistent with diffusion of the cleavage product into the patch.

TABLE 1
Effect of diffusion on fluorescence intensity of
proteolytic products of fluorogenic substrates
(+ = very weak signal; ++ = weak
signal; +++ = well detected signal;
++++ = strong signal;
+++++ = very strong signal).

| Depth of | Diffusion time | | |
|---|---|---|---|
| enzyme (mm) | 30 min | 60 min | 120 min |
| 0 | ++++ | +++++ | +++++ |
| 2 | +++ | ++++ | +++++ |
| 5 | + | ++ | ++++ |

### Example 2

### Detection of Fluorescent Products of Proteolysis in the Extracellular Matrix in Agarose Sandwich Cultures of Human Melanoma Cells Using the “Patch Technique”

Experiments were performed to demonstrate that the patch technique can be used to detect MMPs activity in cultures of human melanoma cells. All in vitro experiments were done in triplicate and were repeated a minimum of two times.

Human melanoma cell lines SK-MEL-28 and WM 266-4 and mouse melanoma cell line B16 were obtained from the American Tissue Culture Collection (ATCC) and were cultured according to recommendations of ATCC (DMEM, 10% FCS, penicillin +streptomycin). Cells were used in experiments after two passages in the laboratory. Confluent cultures in 60-mm Petri dishes were used in these experiments. Liquid culture media were removed and replaced with low melting temperature agarose (Sigma, cell culture grade) in DMEM. After a gel was formed (approximately 1-2 mm thick), a cellulose membrane previously immersed in a solution of high affinity fluorogenic substrates for MMPs, as described in Example 1, was placed on top of the gel. Membranes were removed after 30, 60, 120 and 360 minutes and fluorescence was analyzed as described in Example 1. Fluorescence was detected in patches that were in contact with agar for 120 and 360 minutes, but not in controls.

### Example 3

### In Vivo Detection of MMP Activity in a Mouse Model of Melanoma

Experiments were performed to demonstrate that the inventive method can be used to detect MMP activity in vivo in animal models of melanoma.

Mouse melanoma cell line B16 was used. The B16 cells were cultured and passaged as described above. A total of 5 ×10⁶ cells were injected subcutaneously or intradermally into left and right limbs of three inbred C57BL/6JOIaHsd mice. Four days after injection, when melanoma lesions became very large (about 3-5 mm in diameter), animals were sacrificed, and melanomas with adjacent intact skin were removed. A cellulose membrane patch that was previously immersed in a solution of high affinity fluorogenic substrates for MMPs, as described in Example 1, was placed on the external surface of the removed specimen, and the membrane was covered with an adhesive bandage to affix the membrane patch to the skin for the time period of interest. The membrane patches were removed after 60 and 120 minutes and fluorescence was analyzed, as described in Example 1. Fluorescence was detected in patches that were in contact with melanoma tumor for either 60 minutes or 120 minutes, but not in the controls minus fluorogenic substrate.

In other experiments, mouse melanoma cell line B16 was cultured as described above, and approximately 5 ×10⁶ cells were injected subcutaneously or intradermally into the left and right limbs of three C57BL/6JOIaHsd mice. After 4 days, when melanomas were approximately 2 mm in diameter, the membrane patches, as described in Example 1, were placed directly on top of the skin exhibiting melanoma and affixed with adhesive bandages as described above. Patches were removed after 90 minutes, and fluorescence analyzed. In all cases of melanoma, a fluorescent signal was visualized using UV light.

In still other experiments human melanoma cell lines SK-MEL-28 (ATCC HTB-72) and WM 266-4 (ATCC CRL-1676) were cultured according to recommendations of ATCC (DMEM, 10% FCS, penicillin/streptomycin). 5 ×10⁶ cells (1:1 mixture of both cell lines) were injected subcutaneously or intradermally into the left and right limbs of two Hsd:Athymic-nu mice. Large skin tumors (diameter 5 mm) developed after 10 weeks. The tumors were mobile (i.e., tumors formed a compact mass of cells and were not infiltrating epidermis and muscle) and were completely covered with intact epidermis.

A cellulose membrane patch, as described in Example 1, was placed on the skin with melanoma tumors. In a control experiment, the patch loaded with fluorogenic MMPs substrate was applied to the skin of intact (living) mouse. Patches were removed after 70 minutes and fluorescent signal visualized using UV light. Fluorescent products of proteolysis were detected in patches that covered skin melanomas. In contrast, a test patch applied to healthy skin showed no fluorescence.

### Example 4

### Patch Analysis of Melanomas and Nonmalignant Lesions in Humans

Study included 7 melanoma patients and 6 patients with nonmalignant lesion. Patch analysis was used to compare proteolytic activity of MPPs using fluorogenic peptides in melanoma and nonmalignant lesions.

Materials

Patch: A cellulose membrane

Peptides: Mixture of Two Peptides

1. DABCYL-GABA-Pro-GLN-Gly-Leu-Glu(EDANS)-Ala-Lys-NH (Calbiochem) — before use membrane was soaked in 10⁻⁵ M solution of peptide in 20 mM Tris buffer, pH 7.6, 2.5 mM ZnSO₄ and 5 mM CaCl₂.

2. DABCYL-GABA-Arg-Pro-Lys-Pro-Val-Glu-Nva-Trp-Arg-Glu(EDANS)-Ala-Lys-N H (Calbiochem) — before use membrane was soaked in 10⁻⁵ M solution of peptide in 20 mM Tris buffer, pH 7.6, 2.5 mM ZnSO₄ and 5 mM CaCl₂.

Patch —1 cm² cellulose membrane, added 100 μl of peptide mixture. Applied on skin for 2 hours before surgical removal of lesion. All lesions were on the arm. Cellulose membrane was fixed with a bandaid adhesive.

Patch observed immediately after removal under UV light followed by extraction of peptides using 20 mM Tris buffer, pH 7.6. Fluorescence measured after 1 hour incubation of patch in 1 ml of buffer.

Results Table 2

Analysis of results showed that five out of 7 melanoma patients had detectable fluorescent signal in patch, whereas signal was undetectable in 5 out of six patients with nonmalignant skin lesion.

TABLE 2

| ID | Signal in patch | Fluorescence Intensity |
|---|---|---|
| M1 | Yes | 0.09 |
| M2 | Yes | 3.7 |
| M3 | No | 0 |
| M4 | Yes | 1.3 |
| M5 | No | 0 |
| M6 | Yes | 0.06 |
| M7 | Yes | 0.13 |
| NM1 | No | 0 |
| NM2 | No | 0 |
| NM3 | No | 0 |
| NM4 | Yes | 0.025 |
| NM5 | No | 0 |
| NM6 | No | 0 |
| control | No | 0 |

M—melanoma
NM—nonmalignant lesion
control - cellulose membrane, no exposure to skin

1. A patch for detecting a cancer, comprising:
a carrier layer comprising a pharmaceutically acceptable aqueous solution comprising a first probe that comprises a peptide substrate of a first proteolytic enzyme of interest, the first proteolytic enzyme being an extracellularly secreted enzyme, the activity of which is characteristic of the cancer, wherein after cleavage of the peptide substrate by the first proteolytic enzyme, if present, a detectable first cleavage product is formed; and
wherein the carrier layer comprises a semipermeable surface permeable to the first probe and the first cleavage product, and the semipermeable surface is adapted for being contacted with an externally accessible tissue surface such that the first probe and the first cleavage product can diffuse through the semipermeable surface of the carrier layer.

2. The patch of claim 1, wherein the peptide substrate is an oligopeptide substrate analog of the first proteolytic enzyme.

3. The patch of claim 1, wherein the first cleavage product is detectable by interaction with a specific antibody, aptamer, or nanoparticle.

4. The patch of claim 1, wherein the first probe further comprises a detectable label such that after cleavage of the oligopeptide substrate analog by the first proteolytic enzyme, a first cleavage product is formed that bears the detectable label.

5. The patch of claim 4, wherein the label is a fluorochrome.

6. The patch of claim 4, wherein the label is a radioisotope.

7. The patch of claim 4, wherein the label is a stable isotope.

8. The patch of claim 1, wherein the pharmaceutically acceptable aqueous solution further comprises a second probe that comprises a peptide substrate of a second proteolytic enzyme of interest, the second proteolytic enzyme being an extracellularly secreted enzyme, wherein, after cleavage of the peptide substrate by the second proteolytic enzyme, if present, a detectable second cleavage product is formed; and
wherein the semipermeable surface of the carrier layer is further permeable to the second probe and the second cleavage product.

9. The patch of claim 8, wherein the peptide substrate of the second proteolytic enzyme is an oligopeptide substrate analog of the second proteolytic enzyme.

10. The patch of claim 8, wherein the second cleavage product is detectable by interaction with a specific antibody, aptamer, or nanoparticle.

11. The patch of claim 8, wherein the second probe further comprises a detectable label such that after cleavage of the peptide substrate by the second proteolytic enzyme, a second cleavage product is formed that bears the detectable label.

12. The patch of claim 11, wherein the label is a fluorochrome.

13. The patch of claim 11, wherein the label is a radioisotope.

14. The patch of claim 11, wherein the label is a stable isotope.

15. A patch for detecting a skin cancer, comprising:
a carrier layer comprising a pharmaceutically acceptable aqueous solution comprising an intramolecularly quenched fluorogenic first probe that comprises an oligopeptide substrate analog of a first proteolytic enzyme the proteolytic enzyme being an extracellularly secreted enzyme, the activity of which is characteristic of the skin cancer, wherein the first probe is fluorogenically quenched before cleavage of the oligopeptide substrate analog of the first proteolytic enzyme, and wherein, after cleavage of the oligopeptide substrate analog by the first proteolytic enzyme, if present, a fluorescent first cleavage product is formed that fluoresces at a first wavelength; and
wherein the carrier layer comprises a semipermeable surface permeable to the first probe and the first cleavage product, and the semipermeable surface is capable of being contacted with an externally accessible tissue surface such that the first probe and the first cleavage product can diffuse through the semipermeable surface of the carrier layer.

16. The patch of claim 15, wherein the pharmaceutically acceptable aqueous solution further comprises an intramolecularly quenched fluorogenic second probe that comprises an oligopeptide substrate analog of a second proteolytic enzyme of interest, the second proteolytic enzyme being an extracellularly secreted enzyme, wherein the second probe is fluorogenically quenched before cleavage of the oligopeptide substrate analog of the second proteolytic enzyme, and wherein, after cleavage of the oligopeptide substrate analog by the second proteolytic enzyme, a fluorescent second cleavage product is formed that fluoresces at a second wavelength different from the first wavelength; and
wherein the semipermeable surface of the carrier layer is further permeable to the second probe and the second cleavage product.

17. The patch of any of claims 1, 2, 3, 4, 5, 6, 7, 8, 9, 10, 11, 12, 13, 14, 15, or 16, wherein the first or second proteolytic enzyme, or both, is a serine protease, a cathepsin, or a metalloproteinase.

18. A method of detecting a cancer in a tissue of a mammalian subject, comprising:
(A) topically applying to an externally accessible surface of the tissue the patch of any of claims 1, 2, 3, 4, 5, 6, 7, 8, 9, 10, 11, 12, 13, 14, 15, or 16;
(B) allowing the first probe or the second probe, or both, to diffuse into the tissue such that, if the first proteolytic enzyme or the second proteolytic enzyme, or both, is present in the tissue, the first probe binds to the first proteolytic enzyme, the second probe binds to the second proteolytic enzyme, and enzymatic cleavage of the peptide substrate results, thereby forming the first cleavage product or the second cleavage product, or both; and then
(C) detecting the presence or absence of the first cleavage product or the second cleavage product, or both, by applying suitable detection means, whereby proteolytic activity that is characteristic of the cancer is indicated and the cancer is detected thereby.

19. A method of detecting a skin cancer in a tissue of a mammalian subject, comprising:
(A) topically applying to an externally accessible surface of the tissue the patch of claim 15;
(B) allowing the first probe to diffuse into the tissue such that, if the first proteolytic enzyme is detectably present in the tissue, the first probe binds to the first proteolytic enzyme and enzymatic cleavage of the peptide substrate results, thereby forming the first cleavage product; and then
(C) detecting the presence or absence of fluorescence at the first wavelength, whereby the presence of fluorescence at the first wavelength indicates the activity of the first proteolytic enzyme that is characteristic of the skin cancer, and the skin cancer is detected thereby.

20. The method of claim 18 or claim 19, wherein the externally accessible surface is a mucous membrane.

21. The method of claim 18 or claim 19, wherein the externally accessible surface is epidermis.

22. The method of claim 18 or claim 19, wherein the cancer is a melanoma.

23. The method of claim 18 or claim 19, wherein the first or second proteolytic enzyme, or both, is a serine protease, a cathepsin, or a metalloproteinase.

24. The method of claim 18, wherein detecting the presence or absence of the first cleavage product or of the second cleavage product, or both, is performed by applying detection means to the patch.

25. The method of claim 18, further comprising extracting the cleavage products from the patch to obtain an extract, and applying detection means to the extract.

26. The method of claim 19, wherein detecting the presence or absence of fluorescence is performed by applying fluorescence detection means to the patch.

27. The method of claim 19, further comprising extracting the cleavage products from the patch to obtain an extract, and applying fluorescence detection means to the extract.
